(19) United States
(12) Patent Application Publication
(10) Pub. No.: US 2012/0232385 A1
(43) Pub. Date: Sep. 13, 2012

(54) RADIATION IMAGING DEVICE AND NUCLEAR MEDICINE DIAGNOSTIC DEVICE USING SAME

(75) Inventors: Kaori Hattori, Okayama (JP); Atsuro Suzuki, Hitachi (JP); Katsutoshi Tsuchiya, Hitachi (JP); Takafumi Ishitsu, Hitachi (JP); Keiji Kobashi, Mito (JP)

(73) Assignee: HITACHI, LTD., Tokyo (JP)

(21) Appl. No.: 13/508,753

(22) PCT Filed: Oct. 29, 2010
(86) PCT No.: PCT/JP2010/069286

§ 371 Date: May 9, 2012

(30) Foreign Application Priority Data
Nov. 13, 2009 (JP) 2009-260389

(51) Int. Cl. A61B 6/02

(52) U.S. Cl. 600/436

(57) ABSTRACT

The radiation imaging device acquires incident position information of radiation for each detector by setting the size of a through-hole such that at least one pixel is arrayed in a plane view from a direction perpendicular to the detector plane; arranges a septa in displacing from a boundary line between a detector pair in a plane view from a direction perpendicular to the detector plane; further arranging the septa so as to be orthogonal to the boundary line between the detector pair in a plane view from a direction perpendicular to the detector plane; arranges the top of the through-hole in a plane view from a direction perpendicular to the detector plane, in displacing from the boundary line of the detector pair; and arranges the top of the detectors in a plane view from a direction perpendicular to the detector plane so as to be visible through the through-hole.

### TECHNICAL FIELD

The present invention relates to a radiation imaging device having a pixel-type measurement system and for converting an incident radiation distribution to an image, and a nuclear medicine diagnostic device using the same.

### BACKGROUND ART

As a device applying the radiation measuring device to the nuclear medicine field, there is a Single Photon Emission Computed Tomography device (hereafter referred to as a SPECT device) using a gamma camera. This SPECT device is the one for measuring the distribution of a compound containing a radioactive isotope to provide an image of a tomogram plane.

Main stream of a radiation detector which has been used in the conventional SPECT device has been such one that combines a scintillator constituted of a sheet of large crystal, and a plurality of photomultiplier tubes. In addition, this SPECT device performs position determination of radiation by calculation of center of gravity from output signals of a plurality of photomultiplier tubes.

However, this method has resolution limitation of about 10 mm, which is insufficient to be used in a clinical practice, therefore the SPECT device with higher resolution has been required.

In recent years, as a device having higher resolution, a pixel-type radiation detector (hereafter referred to as a detector) has been developed. The pixel-type detector includes the one constituted of a scintillator and a photodiode, or the one constituted of a semiconductor for converting a radiation to an electric signal, or the like. Any of which acquires a position signal in a small detector unit, that is, in a pixel unit. Therefore, inherent resolution of the detector is determined by a pixel size, and measurement is performed in a spatially discrete way. In addition, a pixel-type detector with a pixel size of about 1 to 2 mm has also been developed, whose resolution has attained 10 mm or smaller, and has thus been improved significantly.

On the other hand, a reconstruction method of a tomography has also been developed and improved, contributing largely to enhancement of resolution. Up to now, there has been used an FBP method (a Filtered Back-Projection method), a iterative reconstruction method without resolution correction (an MLEM method: a Maximum Likelihood Expectation Maximization method), an Ordered Subset Expectation Maximization method (an OSEM method) using a subset, or the like. In recent years, an iterative reconstruction method with resolution correction has been developed. This method is capable of reconstructing an image by considering physical factors such as geographical shape of a collimator or a detector, and scattered radiation, and thus providing a more accurate image.

It should be noted that, in the following explanation on the pixel-type detector, terminologies of “a detector” and “a detector group” will be used, wherein the detector shall be the one constituting one pixel with an arbitrary shape, and the detector group shall be an aggregate where the detectors are arrayed.

In general, a shape of the detector is rectangle, and the detector group provides, when viewed from a radiation incident side, a constitution closely packaged with rectangles.

In all detectors constituting the detector group, to make sensitivity uniform, a through-hole of the collimator and the detector are arranged so as to make one by one response, in many cases. In addition, in view of easy handling, it is general that a shape of the through-hole of the collimator is also rectangle, to match with a shape of the detector.

Here, in the case where the detector is rectangle, one detector contacts with adjacent detectors at four planes. The plane contacting with the adjacent detector shall be defined as “a boundary plane between the detector pair”. In addition, a boundary plane between the detector pair in a plane view from a direction perpendicular to the detector plane shall be defined as “a boundary line between the detector pair”.

In a conventional SPECT device, septa of the collimator has been arranged on the boundary line between this detector pair, in a plane view from a direction perpendicular to the detector plane.

On the other hand, it has been known that miss-positioning of the collimator and the detector causes moiré. To solve this problem, constitutions where the collimator is rotated have been disclosed (PATENT LITERATURE 1). These constitutions are capable of reducing and uniformizing moiré because of keeping constant area of a septa traversing on the detector, even when in miss-positioning of the collimator from a predetermined position.

At present, such a SPECT device has been required in a clinical practice that has high spatial resolution and high sensitivity. There are many factors which determine resolution or sensitivity, such as distance between a radiation source and the detector, thickness of the septa, along with energy, scattering and absorption of radiation.

Among these factors, height of the septa of the collimator and size of the through-hole of the collimator have largely influence on determination of resolution and sensitivity.

That is, to acquire high resolution, it is necessary to limit arrival direction of incident radiation to the detector by the collimator. To attain this, a view of measured object from the detector may be narrowed by the collimator. As such a collimator, there has been known an LEHR (Low Energy High Resolution) collimator. However, this limitation sacrifices sensitivity.

On the other hand, to acquire high sensitivity, it is necessary to increase size of the through-hole of the collimator. As such a collimator, there has been known an LEGP (Low Energy General Purpose) collimator or an LEHS (Low Energy High Sensitivity) collimator. However, increase in size of the through-hole deteriorates resolution.

In this way, because high resolution and high sensitivity cannot be attained at the same time, in a conventional SPECT device, it is necessary to exchange the collimator in response to applications, which forces load at a clinical practice.

Accordingly, as the SPECT device satisfying both of sensitivity and resolution, such a SPECT device has been invented, that contains a plurality of detectors in one rectangular through-hole. This SPECT device has been verified to provide higher resolution than that of a conventional SPECT device having one by one response between the through-hole and the detector, when compared under the same size of the through-hole (PATENT LITERATURE 2, NON-PATENT LITERATURE 1).

### CITATION LIST

### Patent Literature

PATENT LITERATURE 1: JP Patent No. 3928647
PATENT LITERATURE 2: WO 2008/046971

### Non-Patent Literature

NON-PATENT LITERATURE 1: C. Robert et al. (2008) 2008 IEEE Nuclear Science Symposium Conference Record Vol 6 pp. 4246-4251

### SUMMARY OF INVENTION

### Technical Problem

To acquire a uniform image without the non-uniformity of sensitivity or artifact, in the SPECT device, it is important that a shape of the collimator is uniform, and positioning of the collimator and the detector is performed accurately.

In addition, the SPECT device, where a plurality of the detectors are included in one through-hole, and containing a constitution, where a septa of the collimator is arranged so as to be on the boundary line between this detector pair, in a plan view from a perpendicular direction, is required to have higher accuracy in manufacturing precision and positioning of the collimator, as compared with a conventional SPECT device having the through-hole and the detector in one by one response.

In a constitution, where a plurality of the detectors are included in one through-hole, and a septa of the collimator is arranged on the boundary line between this detector pair, in a plan view from a perpendicular direction, influence of miss-positioning of the collimator is more serious. Miss-positioning of the collimator provides not only broad non-uniformity of sensitivity caused by moiré but also a stripe shape periodic pattern. It is because miss-positioning of the collimator leads to such an arrangement that a septa is arranged on a certain detector but a septa is not arranged on a certain other detector. Therefore, miss-positioning of the collimator forms the non-uniformity of sensitivity having a periodic stripe shape. It should be noted that this non-uniformity of sensitivity having a periodic stripe shape is determined by positional relation between the septa and the detector, therefore the period is about several pixels.

It has been known that reconstitution using an image having the non-uniformity of sensitivity having a stripe shape forms ring-artifact. The non-uniformity of sensitivity having a short period generates short period artifact. In this case, a fine structure of a tomography image is lost, thus significantly deteriorating image quality.

Therefore, in the SPECT device, where a plurality of the detectors are included in one rectangular through-hole, more strict positioning of the collimator is required. However, in the present manufacturing precision of the collimator and the positioning method, it is difficult to prevent artifact completely.

It should be noted that this phenomenon does not occur in a conventional SPECT device that the through-hole corresponds one-to-one with the detector. It is because positional relation between all of the detectors and the septa is the same locally, even in miss-positioning of the collimator.

In addition, the collimator to be used in the SPECT device is usually made of lead, and lead is relatively soft and has easy deforming property, and is thus difficult to maintain sufficient manufacturing precision. In addition, large area of the collimator provides easy deflection by own weight of lead.

Additionally, the SPECT device gives, in imaging, complicated movement such as rotation of a camera having the built-in detector and collimator. In this case, there is a problem of miss-positioning of the collimator from predetermined position.

On the other hand, there has been known a method for reconstructing an image by measuring a response function by a point radiation source and using the measured response function. However, it requires measurement of the point radiation source at many positions, which takes time in measuring the response function. As described above, in the SPECT device, various kinds of collimators are exchanged often in use in response to applications. It is not rational that calibration as above is performed each time of exchange. Therefore, such a SPECT device has been required that is robust against miss-positioning of the collimator.

In view of the above circumstance, it is an object of the present invention to provide a radiation imaging device, which is robust (stable) with respect to miss-positioning of a collimator, even when detectors and a collimator having conventional manufacturing precision are used, and a nuclear medicine diagnostic device using same.

### Solution to Problem

To solve such a problem, a radiation imaging device relevant to claim 1 of the present invention is a radiation imaging device having: a pixel-type detector group where detectors are arrayed having a spread as a plane, and each detector constitutes each pixel; a radiation measuring circuit which reads out a detection signal from the detector group; a collimator partitioned by a septa, and arrayed with a plurality of through-holes which are extending in a direction perpendicular to the detector plane; characterized by acquiring incident position information of radiation for each detector by: setting the size of a through-hole such that one or a plurality of pixels are arrayed in a plane view from a direction perpendicular to the detector plane; arranging the septa in displacing from a boundary line between a detector pair in a plane view from a direction perpendicular to the detector plane; further arranging the septa so as to be orthogonal to the boundary line between the detector pair in a plane view from a direction perpendicular to the detector plane; arranging the top of the through-hole in a plane view from a direction perpendicular to the detector plane, in displacing from the boundary line of the detector pair; and arranging the top of the detectors in a plane view from a direction perpendicular to the detector plane so as to be visible through the through-hole.

### Advantageous Effects of Invention

According to the present invention, a radiation imaging device, which is robust (stable) with respect to miss-positioning of a collimator, even when detectors and a collimator having conventional manufacturing precision are used, and a nuclear medicine diagnostic device using same can be provided.

Other objectives, features and advantages of the present invention will be made clear from the following Examples of the present invention with reference to the attached drawings.

### BRIEF DESCRIPTION OF THE DRAWINGS

FIG. 1 is a constitutional drawing of a SPECT device relevant to an embodiment of the present invention.

FIG. 2 is a perspective view showing a pixel-type detector to be built-in to a camera relevant to an embodiment of the present invention.

FIG. 3 is a perspective view showing another example of a pixel-type detector.

FIG. 4 is a perspective view showing an incident plane side of a first modification example of another pixel-type detector.

FIG. 5 is a perspective view showing an opposite side of an incident plane side of a first modification example of another pixel-type detector.

FIG. 6 is a perspective view showing a second modification example of another pixel-type detector.

FIG. 7 is a perspective view showing a pixel-type scintillator detector.

FIG. 8 is a perspective view showing arrangement of collimators and detectors relevant to an embodiment of the present invention.

FIG. 9 is a perspective view showing arrangement of collimators and detectors relevant to Comparative Example.

FIG. 10 is arrangement of collimators and detectors relevant to Comparative Example, viewed from a radiation irradiation direction.

FIG. 11 is arrangement of collimators and detectors relevant to an embodiment of the present invention, viewed from a radiation irradiation direction.

FIG. 12 is a drawing showing an arrangement example of a pixel-type detector and a septa of a collimator.

FIG. 13 is a drawing showing an arrangement example of a pixel-type detector and a septa of a collimator.

FIG. 14 is a drawing showing an arrangement example of a pixel-type detector and a septa of a collimator.

FIG. 15 is a cross-sectional schematic view explaining relation between a collimator and a detector relevant to Comparative Example and leaked radiation.

FIG. 16 is a cross-sectional schematic view explaining relation between a collimator and a detector relevant to an embodiment of the present invention and leaked radiation.

FIG. 17A is an imaging simulation result of a constitution using a collimator relevant to Comparative Example.

FIG. 17B is an imaging simulation result of a constitution using a collimator relevant to Comparative Example.

FIG. 17C is an imaging simulation result of a constitution using a collimator relevant to Comparative Example.

FIG. 18A is an imaging simulation result of a constitution using a collimator relevant to an embodiment of the present invention.

FIG. 18B is an imaging simulation result of a constitution using a collimator relevant to an embodiment of the present invention.

FIG. 18C is an imaging simulation result of a constitution using a collimator relevant to an embodiment of the present invention.

FIG. 19 is a drawing showing change of imaging simulation result by miss-positioning of a collimator in a constitution using a collimator relevant to Comparative Example.

FIG. 20 is a drawing showing change of imaging simulation result by miss-positioning of a collimator in a constitution using a collimator relevant to an embodiment of the present invention.

FIG. 21 is a graph showing amount of change of a planar source image in a constitution using a collimator relevant to Comparative Example.

FIG. 22 is a graph showing amount of change of a planar source image in a constitution using a collimator relevant to an embodiment of the present invention.

FIG. 23 is a graph showing amount of change of planar source image in Δl/d=4.3, in a constitution using the collimator relevant to an embodiment of the present invention, and a constitution using the collimator relevant to Comparative Example.

FIG. 24 is an example of simulation result of image reconstitution in a constitution using the collimator relevant to an embodiment of the present invention.

FIG. 25 is an example of simulation result of image reconstitution in a constitution using the collimator relevant to Comparative Example.

FIG. 26 is a drawing showing change of simulation result of image reconstitution by miss-positioning of a collimator in a constitution using the collimator relevant to an embodiment of the present invention.

FIG. 27 is a drawing showing change of simulation result of image reconstitution by miss-positioning of a collimator in a constitution using the collimator relevant to Comparative Example.

FIG. 28 is a drawing showing another arrangement of a collimator and a detector relevant to an embodiment of the present invention.

FIG. 29 is a drawing showing another arrangement of a collimator and a detector relevant to an embodiment of the present invention.

FIG. 30 is a drawing showing another arrangement of a collimator and a detector relevant to an embodiment of the present invention.

FIG. 31 is a drawing showing another arrangement of a collimator and a detector relevant to an embodiment of the present invention.

### DESCRIPTION OF EMBODIMENTS

Explanation will be given below in detail on aspect to perform the present invention (hereafter referred to as “embodiment”) with reference to drawings, as appropriate.

### <The SPECT Device (Nuclear Medicine Diagnostic Device) 1>

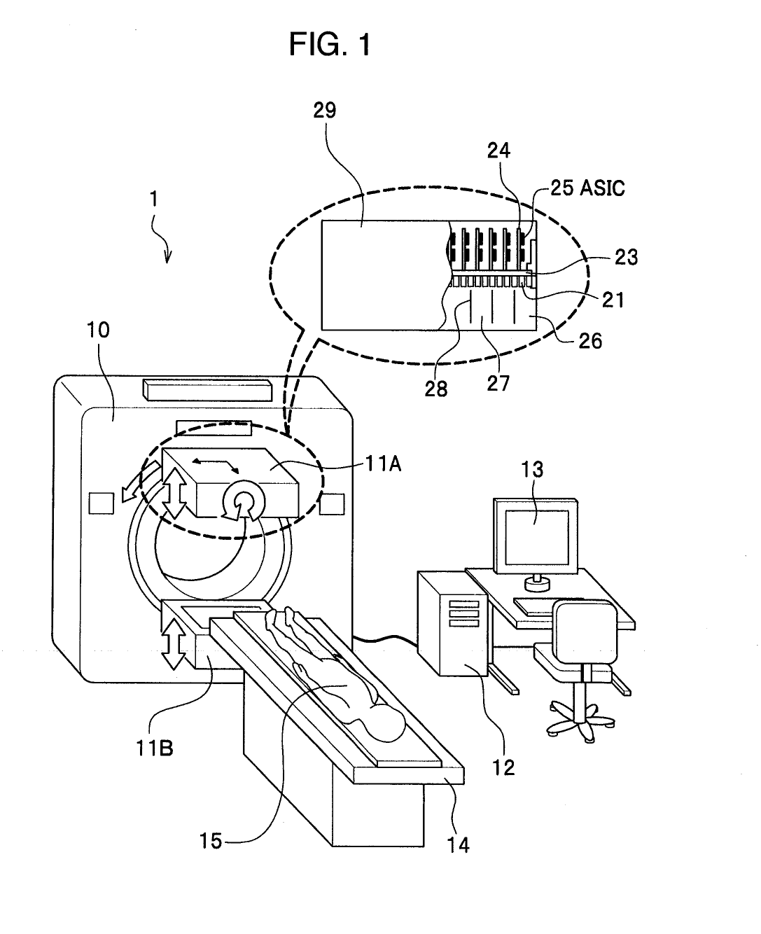

Explanation will be given on total constitution of the SPECT device (nuclear medicine diagnostic device) 1 relevant to an embodiment of the present invention, with reference to FIG. 1.

FIG. 1 is a constitutional drawing of a SPECT device relevant to an embodiment of the present invention.

The SPECT device 1 is constituted by containing a gantry 10, cameras (radiation imaging devices) 11A, 11B, a data processing device 12, a display device 13 and a bed 14.

A subject 15 is administered with a radiopharmaceutical, for example, a radiopharmaceutical containing ^{99m}Tc having a half life of 6 hours. A γ-radiation (radiation) emitted from ^{99m}Tc in a body of the subject 15 mounted on the bed 14 is detected using the camera 11 (11A, 11B) supported on the gantry 10 for imaging a tomography image.

The camera 11 has a collimator 26 and many detectors 21 built-in. The collimator 26 has a through-hole 27 and a septa 28 for partitioning the through-hole 27, and has a role of selecting (restricting incident angle of) the γ-radiation emitted from ^{99m}Tc in a body of the subject 15, and making pass through the γ-radiation only of a certain direction. The γ-radiation passed through the collimator 26 (through-hole 27) is detected using the detector 21.

The camera 11 is provided with an Application Specific Integrated Circuit (hereafter referred to as ASIC) (radiation measuring circuit) 25 for measuring a detection signal of the γ-radiation. As for the detection signal of the γ-radiation, ID of the detector 21 which detected the γ-radiation, as well as wave height value or detection time of the detected radiation are input to an ASIC 25 via a detector substrate 23 and an ASIC substrate 24. They are surrounded by a light shielding•γ-radiation•electromagnetic shield 29 made of iron, lead or the like, constituting the camera 11 to shield light, γ-radiation, and electromagnetic shield.

The camera 11 can be moved in a radial direction and a peripheral direction of the center axis of a cylinder-like opening part, installed at the center part of the gantry 10. In imaging, the camera 11 performs imaging by drawing a nearest-neighbor orbit around the subject 15. In addition, the camera 11 can also be rotated around an attachment part (not shown) to the gantry 10, as the axis, or also made to perform imaging a STATIC image by aligning and fixing two cameras 11A, 11B.

The data processing device 12 has a memory device (not shown) and a tomography image information preparation device (not shown). The data processing device 12 takes in a packet data including data of wave height value and detection time of the detected y-radiation, and ID of the detector (a channel), from the ASIC 25, to form a plane image, or form tomography image information by converting it to sinogram data, and displays it onto the display device 13.

In the case of reconstructing an image in the data processing device 12, there may be the case of using a response function of the detector 21. The response function is a detection probability of a γ-radiation by a certain detector 21, with respect to the y-radiation emitted from a certain region. In general, the response function takes into consideration not only a geometrical shape but also physical factors such as property of scattering, absorption. Use of this response function makes possible to reconstitute a more accurate image using the iterative reconstruction method (MLEM method, OSEM method) or the like. It should be noted that reconstitution of an image using a not correct response function may decrease resolution or form artifact.

In general, in performing tomography imaging, a plurality of planer images are acquired by changing rotation angle to a subject. When the detector group 21A takes a certain angle with respect to a measurement object, count number yᵢ of the detector i satisfies:

yᵢ=ΣCᵢⱼλⱼ

wherein λⱼ represents count number of a detected pixel j. From the above equation, an image is reconstructed using the iterative reconstruction method or the like (an MLEM method, an OSEM method, a MAP method or the like). Here, Cᵢⱼ is a constant determined in a geographical way.

In this way, the SPECT device 1 performs imaging of a radiopharmaceutical accumulated at a tumor or the like in the body of the subject 15 to identify position of the tumor.

### <The Detector 21>

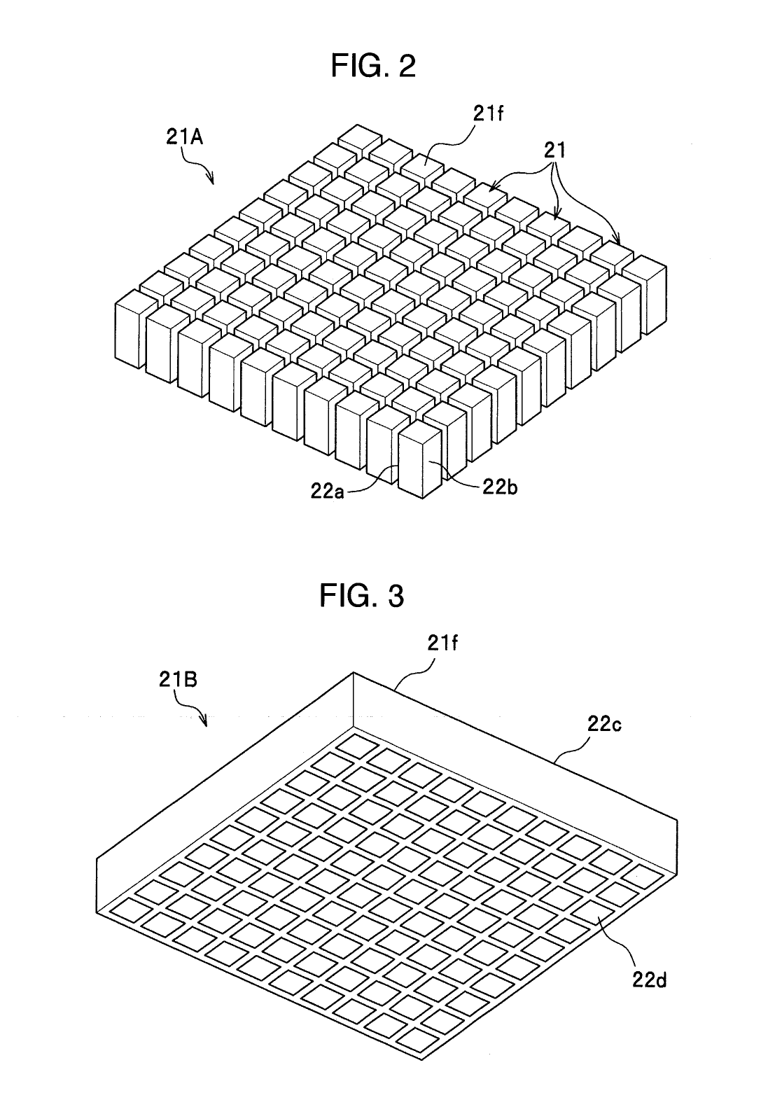

Explanation will be given next on the detector 21 used in the camera 11, with reference to FIG. 2.

FIG. 2 is a perspective view showing a pixel-type detector to be built-in to a camera relevant to an embodiment of the present invention.

The detector group 21A is constituted by arraying the detector 21 using a CdTe semiconductor in two-dimension on a detector substrate 23 (refer to FIG. 1). In addition, each detector 21 constitutes one pixel.

In FIG. 2, the upper face side is an incident plane 21f of the detector 21, and electrodes 22a, 22b for applying voltage are arranged at the side of the detector 21. In this way, different from a scintillator constituted of a single sheet of large crystal, the detection signal is collected in each detector 21 unit, that is, in pixel unit.

It should be noted that, as a matter of convenience to determine the response function, it is desirable that the detector group 21A has a periodic structure. If this is not the case, the response function will be determined pixel by pixel.

It should be noted that detector 21 (the detector group 21A) to be used in the camera 11 is not limited to the one partitioned by each pixel, as shown in FIG. 2, and the detector (a detector group 21B, 21C, 21D, 21E), as shown in FIG. 3 to FIG. 7, may also be used.

FIG. 3 is a perspective view showing another example of a pixel-type detector.

The detector (the detector group 21B), shown in FIG. 3, is the one arranged with a common electrode 22c at one face of a substrate of a CdTe semiconductor, that is, at the whole face of the incident plane 21f side with respect to a substrate of a sheet of the CdTe semiconductor, and arranged with an electrode 22d, partitioned by pixel unit, at the opposite side of the incident plane 21f, where each constitutes a detector corresponding to a pixel, at the substrate of the CdTe semiconductor of an area part equivalent to one piece of the electrode 22d, and the common electrode 22c.

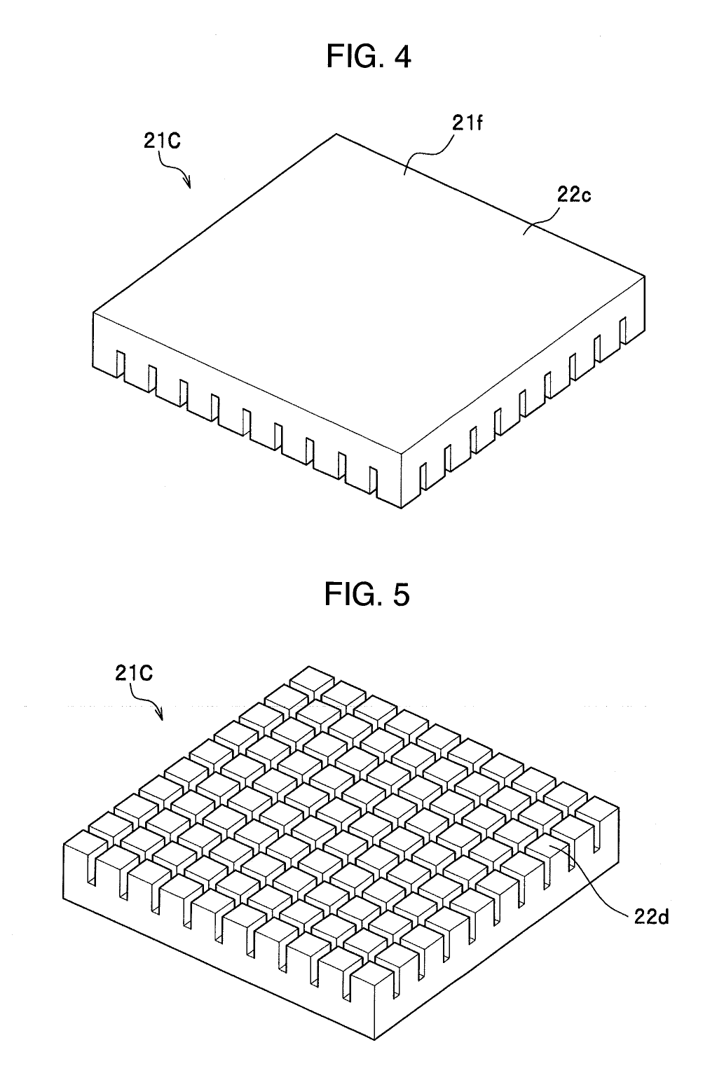

Next, a modification example of the detector (the detector group 21B), shown in FIG. 3, will be shown in FIG. 4 to FIG. 6.

FIG. 4 is a perspective view showing an incident plane side of a first modification example of another pixel-type detector, and FIG. 5 is a perspective view showing an opposite side of an incident plane side of a first modification example of another pixel-type detector.

The detector (the detector group 21C), shown in FIG. 4 and FIG. 5, has a structure arranged with the common electrode 22c at the whole face of the incident plane 21f side with respect to the substrate of a sheet of the CdTe semiconductor, and arranged with an electrode 22d, partitioned by pixel unit, at the opposite side of the incident plane 21f of the substrate of a sheet of the CdTe semiconductor, in addition, where each detector is partitioned by a groove formed by a dicing.

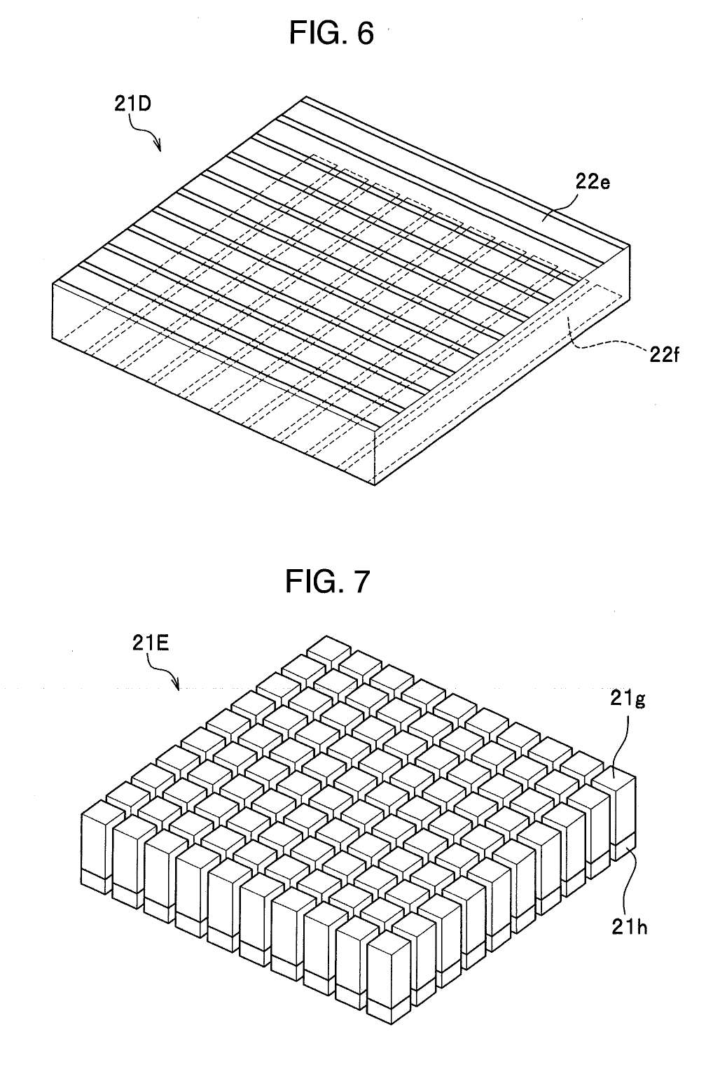

FIG. 6 is a perspective view showing a second modification example of another pixel-type detector.

The detector (the detector group 21D), shown in FIG. 6, is arranged with a plurality of band-like electrodes 22e, 22f in a opposing way, in a right-angle twisted relation, at the upper face and the lower face of a substrate of the CdTe semiconductor, with respect to a substrate of a sheet of the CdTe semiconductor. The band-like electrode 22e at either of the upper face and the lower face is adopted as a positive electrode, and the band-like electrode 22f at the other face is adopted as a negative electrode. A crossing part of the electrode 22e of the positive electrode and the electrode 22f of the negative electrode forms one detector (refer to JP-A-2004-125757).

FIG. 7 is a perspective view showing a pixel-type scintillator detector.

In addition, a structure of the detector may be, like the detector (the detector group 21E) shown in FIG. 7, a scintillator detector constituted of a scintillator 21g and a photodiode 21h, partitioned in pixel unit.

In this case, the side of each scintillator 21g is surrounded by a not shown light shielding material. In addition, as a modification of the scintillator detector shown in FIG. 7, the one constituted of the scintillator 21g partitioned by each pixel, and a Position-Sensitive Photomultiplier Tube (PSPMT) may be adopted.

### <The Collimator 26>

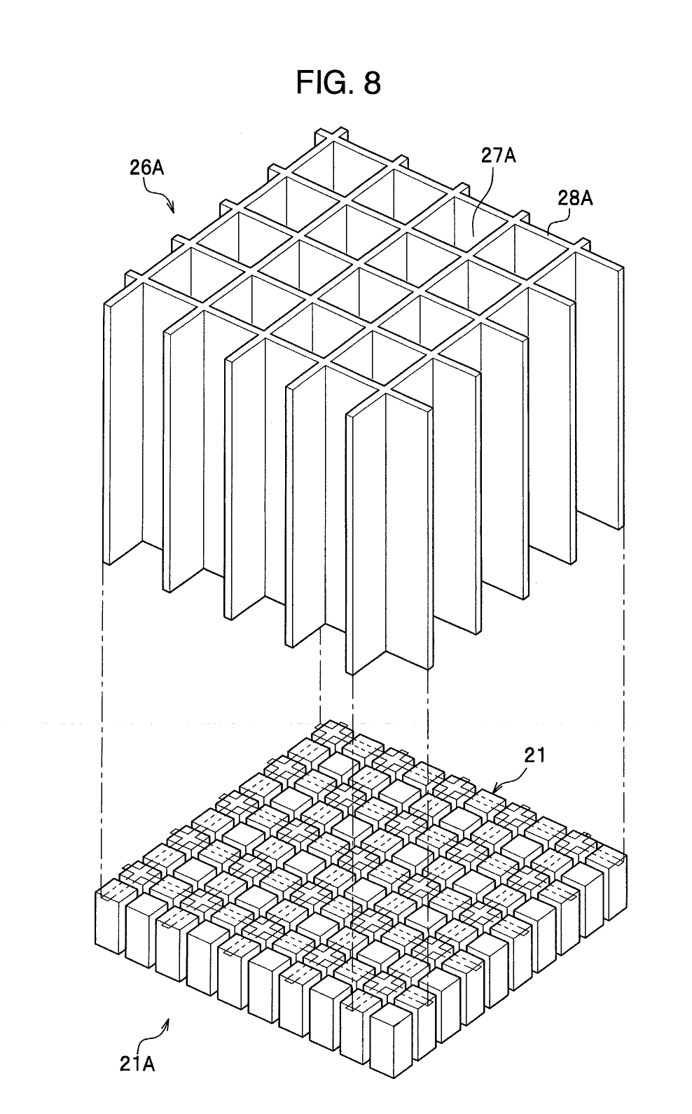

Explanation will be given next on the collimator 26 to be used in the camera 11, specifically, the collimator 26A relevant to an embodiment of the present invention, and the collimator 26B relevant to Comparative Example, with reference to FIG. 8 to FIG. 11.

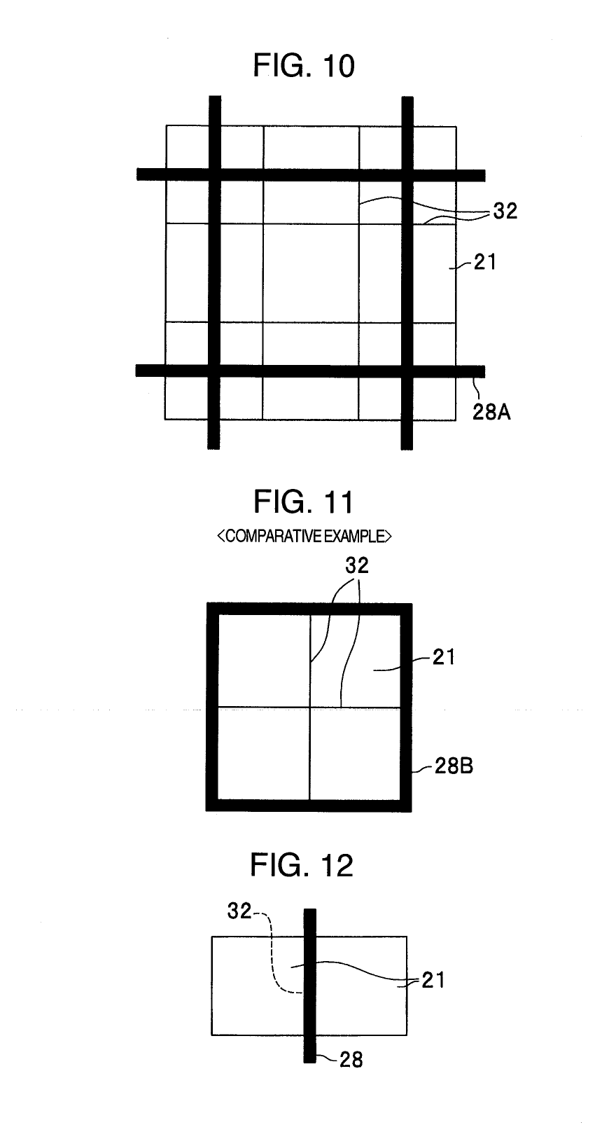

Firstly, explanation will be given on the collimator 26A relevant to an embodiment of the present invention, with reference to FIG. 8 to FIG. 10. FIG. 8 is a perspective view showing arrangement of collimators and detectors relevant to an embodiment of the present invention. FIG. 10 is arrangement of collimators and detectors relevant to Comparative Example, viewed from a radiation irradiation direction.

The collimator 26A is made of lead, and has the through-hole 27A in a view-through direction, in a plane view from a direction perpendicular to the detector plane, where the through-hole 27A is arranged in a square pattern. In addition, each through-hole 27A is partitioned by the septa 28A.

In addition, as shown in FIG. 10, it makes a constitution where M pieces of the detectors 21 are contained relative to one through-hole 27A (in FIG. 10, the constitution case where four pieces of the detectors 21 are contained relative to one through-hole 27A is shown). It should be noted that M may not be an integer.

The septa 28A of the collimator 26A relevant to an embodiment of the present invention, as shown in FIG. 10, is arranged so as to pass through the center of the detector 21. In addition, in the collimator 26A, in a plane view from a direction perpendicular to the detector plane, the boundary line 32 between this detector 21 pair and the septa 28A cross at right angles. This constitution provides small change of count number of radiation of the detector 21, because leaked radiation or shadow of the septa 28A is restricted within nearly the same pixel, even in miss-positioning of the collimator 26A.

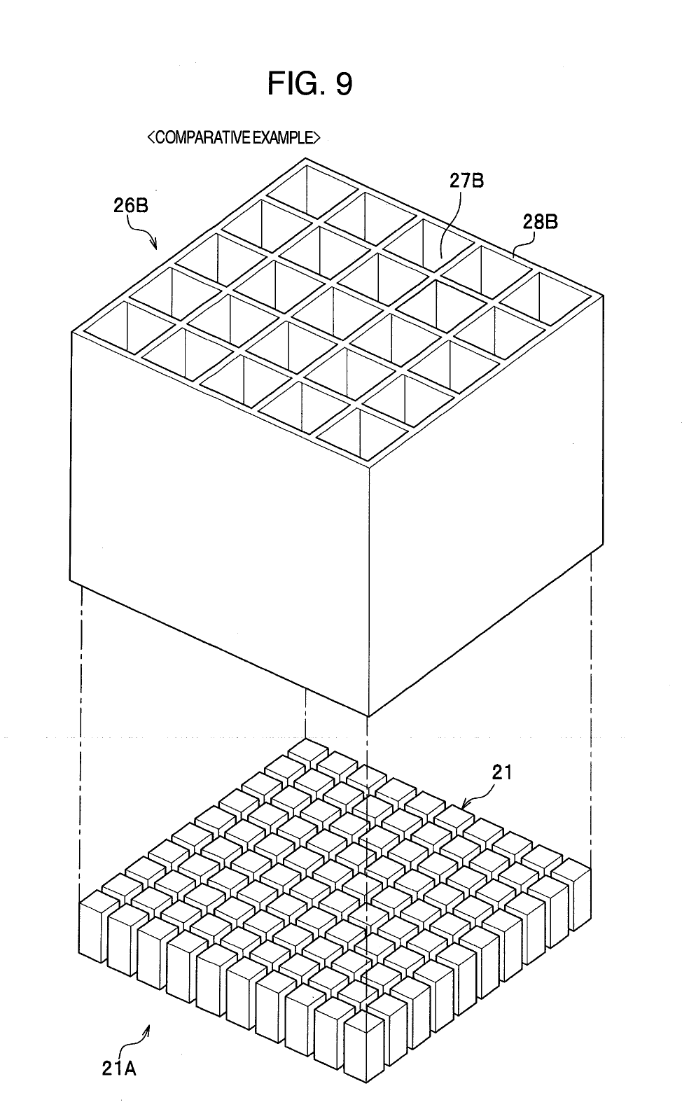

Explanation will be given next on the collimator 26B relevant to Comparative Example, with reference to FIG. 9 and FIG. 11. FIG. 9 is a perspective view showing arrangement of collimators and detectors relevant to Comparative Example. FIG. 11 is arrangement of collimators and detectors relevant to an embodiment of the present invention, viewed from a radiation irradiation direction.

The collimator 26B is made of lead, and has the through-hole 27B, where the through-hole 27B is arranged in a square pattern. In addition, each through-hole 27B is partitioned by the septa 28B.

In addition, as shown in FIG. 11, it is a constitution where M pieces of the detectors 21 are contained relative to one through-hole 27B (in FIG. 10, the constitution case where four pieces of the detectors 21 are contained relative to one through-hole 27B is shown). It should be noted that M may not be an integer.

In this way, the collimator 26A relevant to an embodiment of the present invention and the collimator 26B relevant to Comparative Example have the same constitution except in that arrangement with respect to the detector 21 (the detector group 21A) is different.

The septa 28B of the collimator 26B relevant to Comparative Example, as shown in FIG. 11, is arranged on the boundary line 32 between this detector 21 pair, in a plane view from a direction perpendicular to the detector plane. In this constitution, a short period ring-artifact appears, as will be described later, in miss-positioning of the collimator 26B.

Explanation will be given here on relation between miss-positioning of the collimator 26B and the response function.

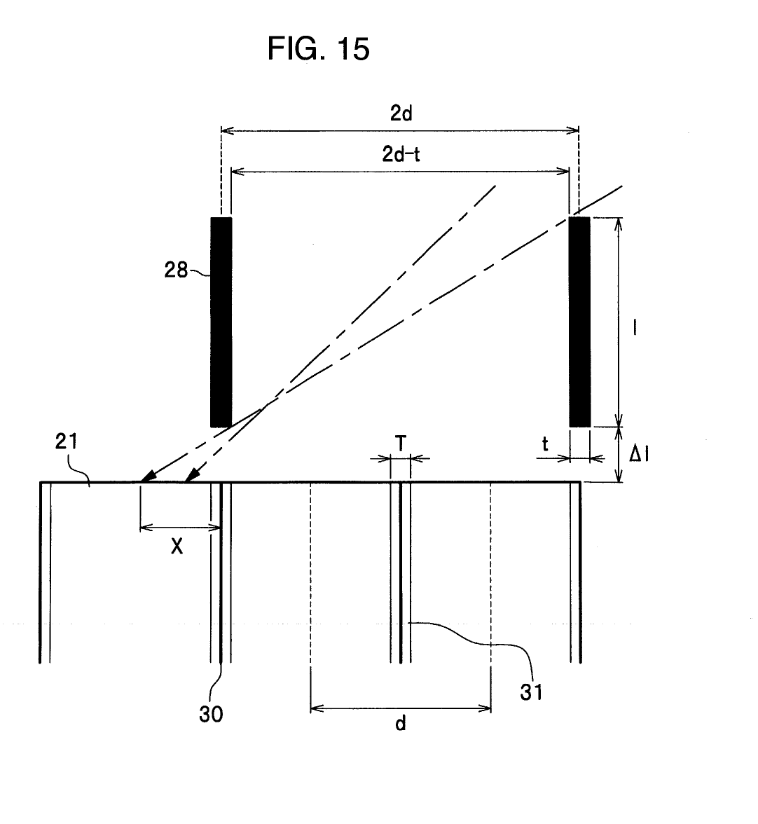

Miss-positioning of the collimator 26B changes the response function. The reason for that is because shadow due to the collimator 26 is observed at the peripheral of the septa 28. In addition, distance between the detector 21 and the collimator 26 cannot be made 0 due to physical restriction. Therefore, as will be shown in FIG. 15 to be described later, leaked radiation from an adjacent pixel is observed. Leaked radiation is detected by the detector 21 positioned at the vicinity of the septa 28. In this way, when position of the collimator 26 moves, position of shadow of the collimator 26 or leaked radiation distribution also moves accompanying with the collimator 26.

By the above reason, radiation distribution at the vicinity of the septa 28 depends strongly on position of the collimator 26. When radiation distribution changes, count number of radiation detected by the detector 21 and the response function change.

As shown in FIG. 12, in the case of containing a constitution where different detectors 21 are arranged sandwiching the septa 28, that is, when the septa 28 is arranged on the boundary line 32 between this detector 21 pair, in a plane view from a direction perpendicular to the detector plane, the response function changes largely with respect to miss-positioning of the collimator 26.

Here, when M is larger than 1, such non-uniformity of sensitivity is created that the septa 28 is positioned on a certain detector, but not present on another detector 21, in miss-positioning of the collimator 26, and an acquired image and the response function change. This non-uniformity of sensitivity appears as a periodic stream. It should be noted that the non-uniformity of sensitivity is determined by positional relation between the septa 28 and the detector 21, and thus gives a short period of about several pixels.

In general, in tomography imaging, a plurality of planar images are acquired by changing a rotation angle around a subject. It has been known that, in the case where a constant stripe shape pattern appears in the planer image irrespective of the angle, ring-artifact appears in a reconstructed image. In the above case, because the non-uniformity of sensitivity is a short period one, a short period ring-artifact appears. The short period artifact erases a fine structure of a tomography image, causing to deteriorate image quality in a large degree.

In this case, even by image image reconstruction (the FBP method or the like) without using the response function, ring-artifact appears. It is because a periodic pattern remains as a periodic pattern, even after reconstitution, to provide artifact. In addition, even in the case of reconstruction using the response function in “no miss-positioning” of the collimator 26, the short period artifact appears. It is because the response function in “no miss-positioning” does not reproduce the periodic pattern, and cannot be corrected either.

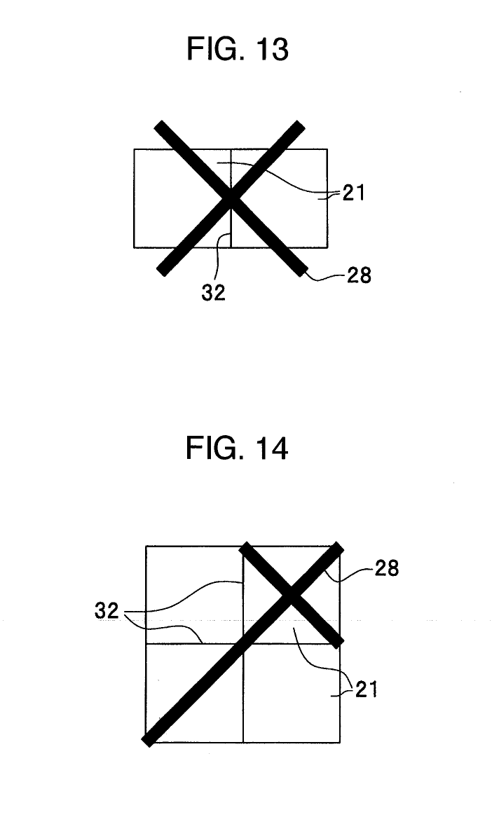

In addition, as shown in FIG. 13, in the case of containing a constitution where a crossing point of the septa 28 (top of the through-hole 27) is present on the boundary line 32 between this detector 21 pair and at the vicinity thereof, in a plane view from a direction perpendicular to the detector plane, artifact is formed by miss-positioning of the collimator 26, due to similar reason as above.

In addition, as shown in FIG. 14, also in the detector 21, which contains a constitution where the top of the detector 21 crosses with and comes close to the septa 28, in a plane view from a direction perpendicular to the detector plane artifact is formed by miss-positioning of the collimator 26, due to similar reason as above.

It should be noted that, in FIG. 13 and FIG. 14, the septa 28 is expressed by a straight line, however, it may be curved.

When M≧1, there has been known a constitution (layout) in which the collimator 26 is arranged in miss-positioned, while maintaining constant area of the septa 28 traversing over the detector 21, to prevent moiré (refer to PATENT LITERATURE 1). Therefore, area for leaked radiation to distribute on one detector 21 is the same irrespective of position of the collimator 26. However, these constitutions (layouts) may partially contain the constitution of FIG. 12, FIG. 13 and FIG. 14.

As shown in FIG. 2 to FIG. 7, a general pixel-type detector 21 has an insensible field 31 between the detector 21 and the detector 21 (refer to FIG. 15 to be described later). Therefore, when the constitution of FIG. 12, FIG. 13 and FIG. 14 is present, even a trace miss-positioning of the collimator 26 changes amount of leaked radiation distributing on the insensible field 31. As a result, count number of radiation of the detector 21 changes. That is, moiré due to miss-positioning and rotation of the collimator 26 cannot be prevented completely.

As described above, such a constitution is good that shadow of the collimator 26 and leaked radiation are included as much as possible in the same detector, even in miss-positioning of the collimator 26. Therefore, like an arrangement of the collimator 26B relevant to Comparative Example and the detector 21, the septa 28 should not come close on to the boundary line 32 between this detector 21 pair, in a plane view from a direction perpendicular to the detector plane.

In addition, the crossing point of the septa 28 (top of the through-hole 27) should not come close onto the boundary line 32 between this detector 21 pair. In addition, the top of the detector 21 should not come close to the septa 28.

Explanation will be given here again, with reference to the collimator 26B relevant to Comparative Example (refer to FIG. 9 and FIG. 11).

Arrangement constitution of the collimator 26B relevant to Comparative Example and the detector 21 (the detector group 21A) contains the constitution shown in FIG. 12, and is unstable with respect to miss-positioning of the collimator 26B.

FIG. 15 is a cross-sectional schematic view explaining relation between a collimator and a detector relevant to Comparative Example and leaked radiation.

Provided that height of the collimator 26 is l, distance between the collimator 26 and the detector 21 is Δl, thickness of the septa 28 is t, distance from the center of the detector 21 to the center of the adjacent detector 21 is d, and distance from the center of one septa 28 to the center of the next septa 28 (that is, a pitch of the septa 28, and a pitch of the through-hole 27) is Nd (it should be noted that N is not limited to an integer), maximum value X of the distance between a position where leaked radiation reaches and the septa 28 is represented by:

|X|=(Nd−t)Δl/l−t/2

In the collimator 26A relevant to an embodiment of the present invention and the collimator 26B relevant to Comparative Example, N=2. In the case of setting at l/d=16, Δl/d=4.3, and t/d=0.3, a range where leaked radiation reaches is about 30% of the size of the detector 21 (that is, |X|/d is about 0.3), whereas it becomes about |X|=0.3 for the detector 21 with d=1 mm.

FIG. 16 is a cross-sectional schematic view explaining relation between a collimator and a detector relevant to an embodiment of the present invention and leaked radiation.

Therefore, in the constitution using the collimator 26A relevant to an embodiment of the present invention (refer to FIG. 8 and FIG. 10), when miss-positioning ΔX of the collimator 26A is in a direction orthogonal to the septa 28A, in a plane view from a direction perpendicular to the detector plane, and ΔX≦d/2−|X|, that is, ΔX≦(d+t)/2−(Nd−t)Δl/l, leaked radiation becomes restricted within the same pixel, and count number of leaked radiation becomes constant.

In addition, the response function is stable with respect to miss-positioning of the collimator 26A within the above range. Although leaked radiation is concentrated at the vicinity of the septa 28, shadow of the collimator 26 distributes widely in the whole pixel. Therefore distribution of the leaked radiation is most influenced by miss-positioning of the collimator 26.

In the SPECT device 1 provided with the collimator 26, having a rectangular detector 21 and a rectangular through-hole 27, when there is space with a distance of (Nd−t)Δl/l−t/2 or more, between the boundary line 32 between this detector 21 pair and the septa 28, which is parallel to this boundary line 32, in a plane view from a direction perpendicular to the detector plane, leaked radiation, which distributes at the vicinity of the septa 28, becomes restricted within the same pixel.

When the insensible field 31 is present between the detector 21 and the detector 21, and provided that width thereof is T, it is necessary to take space with a distance of (Nd−t)Δl/l−t/2+T/2 or more. In addition, in the constitution using the collimator 26A relevant to an embodiment of the present invention, when miss-positioning ΔX of the collimator 26A is in a direction perpendicular to a plane of the septa 28A, and ΔX≦(d+t−T)/2−(Nd−t)Δl/l, leaked radiation becomes restricted within the same pixel, and count number of leaked radiation becomes constant.

It should be noted that, even in trace miss-positioning of the collimator 26 in a direction parallel to the septa 28, count number of the leaked radiation is constant, because reaching area of the leaked radiation counted by the detector 21 is constant.

On the other hand, in the constitution using the collimator 26B relevant to Comparative Example, because |X| is about 0.3 mm, in the detector 21 with d=1 mm, it is understood that miss-positioning of the collimator 26 of an order of 0.1 mm extremely changes amount of leaked radiation detected at one pixel. It is very difficult to perform positioning in a precision of 0.1 mm or smaller. In addition, it is also difficult to prepare the collimator 26 made of lead in this precision.

### <Imaging Simulation in the Case of Irradiation of a Planar Source>

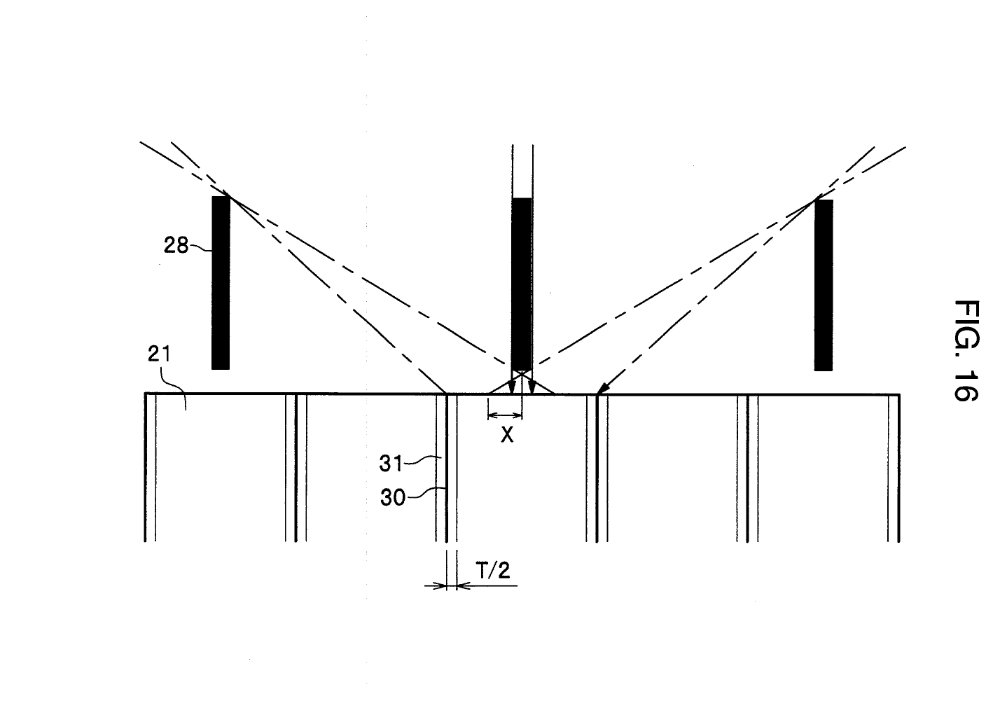

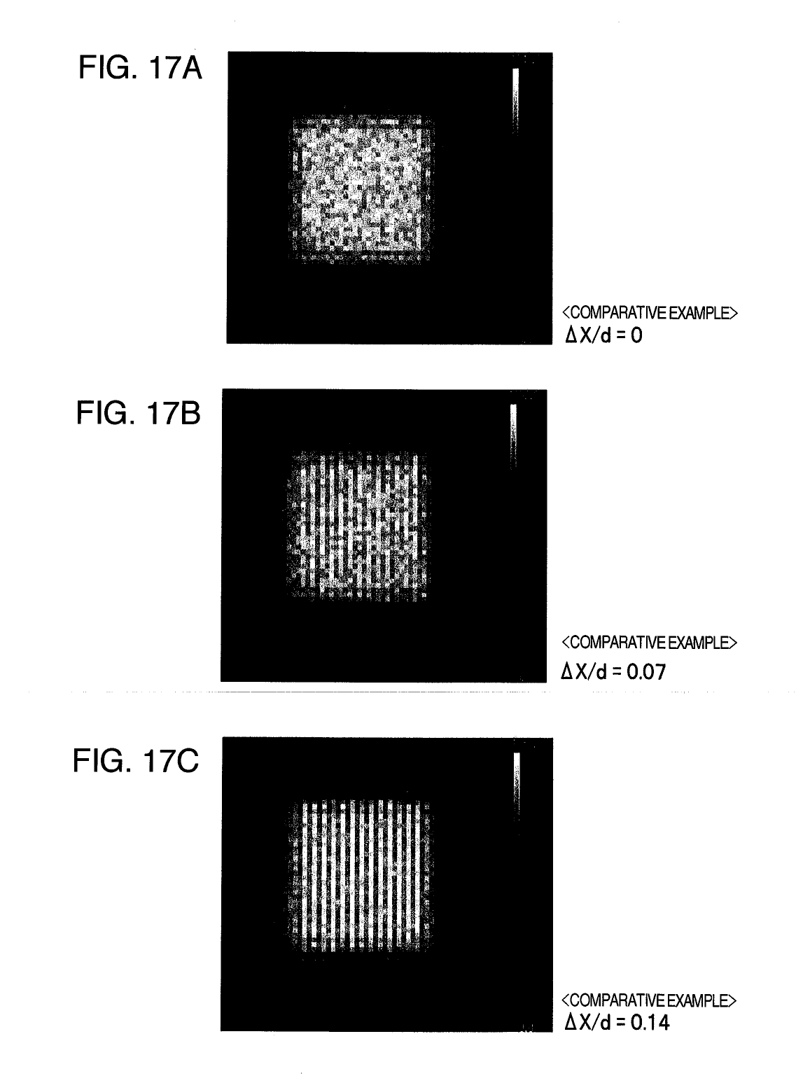

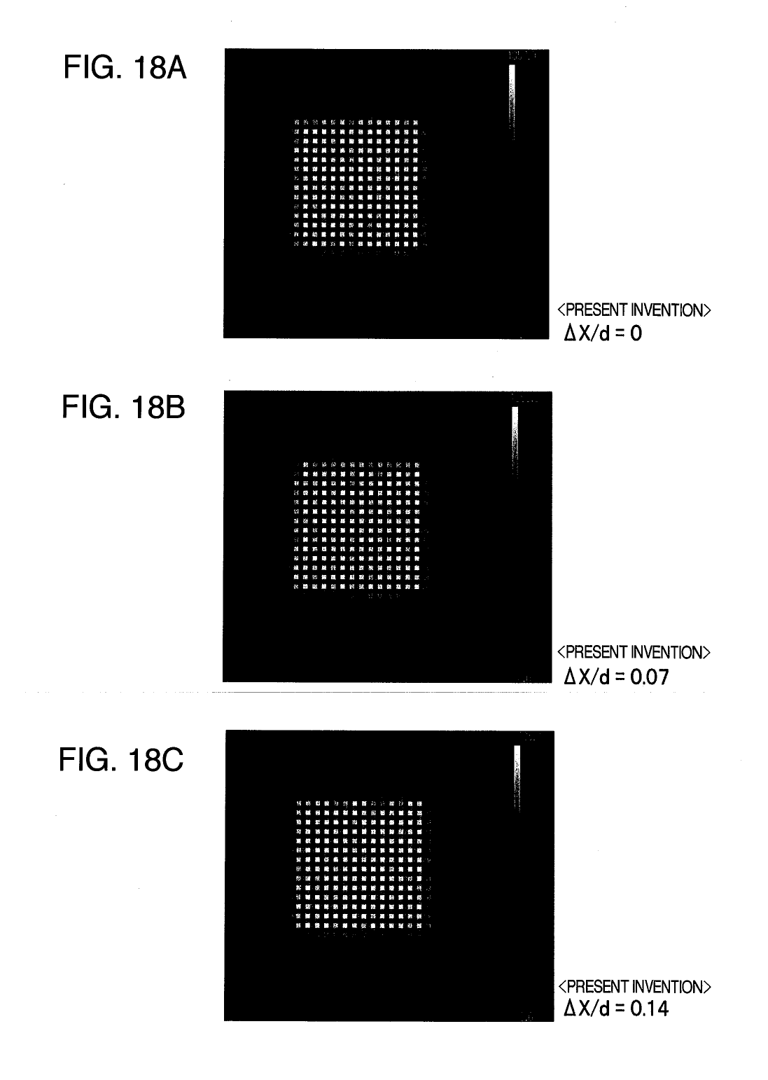

As described above, miss-positioning of the collimator 26 from the predetermined position changes an acquired image. As for the constitution using the collimator 26B relevant to Comparative Example (refer to FIG. 9 and FIG. 11) and the constitution using the collimator 26A relevant to an embodiment of the present invention (refer to FIG. 8 and FIG. 10), images obtained by a simulation using a Monte Carlo method, in the case of irradiation of a uniform planar source, are shown in FIGS. 17A to 17C, and FIGS. 18A to 18C.

FIG. 17A to 17C are imaging simulation results of a constitution using a collimator relevant to Comparative Example and FIG. 18A to 18C are imaging simulation results of a constitution using a collimator relevant to an embodiment of the present invention.

It should be noted that the simulation using the Monte Carlo method was performed by setting position of the planar source at 50 mm from the surface of the collimator 26, l=26 mm, Δl=6 mm, d=1.4 mm, t=0.4 mm, and T=0.1 mm.

In addition, miss-positioning of the collimator 26 from the predetermined position is defined as ΔX.

In addition, in FIGS. 17A to 17C, and FIGS. 18A to 18C, FIG. 17A and FIG. 18A show the case where the collimator 26 is at the predetermined position (ΔX/d=0), FIG. 17A and FIG. 18B show the case where the collimator 26 is miss-positioned from the predetermined position by ΔX/d=0.07 in the right direction, and FIG. 17C and FIG. 18C show the case where the collimator 26 is miss-positioned from the predetermined position by ΔX/d=0.14 in the right direction.

It should be noted that ΔX/d=0.07 is equivalent to ΔX of about 0.1 mm when d=1.4 mm, and ΔX/d=0.14 is equivalent to ΔX of about 0.2 mm when d=1.4 mm.

In the constitution using the collimator 26B relevant to Comparative Example, in the case where the collimator 26B is at the predetermined position (ΔX=0), a uniform image is obtained, as shown in FIG. 17A. However, in the case where the collimator 26B is miss-positioned, even if only by a trance amount, as shown in FIGS. 17B and 17C, a stripe appears in every other column.

On the other hand, in the constitution using the collimator 26A relevant to an embodiment of the present invention, a lattice stripe appears as shown in FIGS. 18A, 18B and 18C.

This is because count number of radiation differs between the detector 21 arranged with the septa 28 at the upward of the detector 21, and the detector 21 not arranged with the septa 28. Therefore, distance from the upper end of the collimator 26 and the planar source does not influence on position of the lattice stripe.

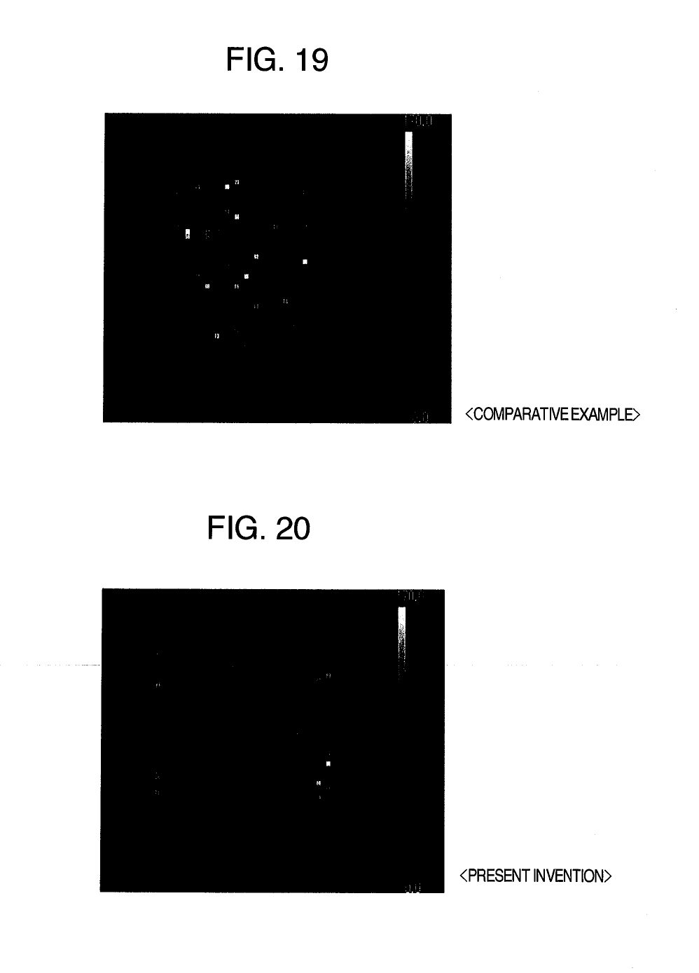

In the constitution using the collimator 26B relevant to Comparative Example and the constitution using the collimator 26A relevant to an embodiment of the present invention, the one obtained by dividing the simulation result in ΔX/d=0.14 by the simulation result in ΔX/d=0, is each shown in FIG. 19 and FIG. 20.

FIG. 19 is a drawing showing change of imaging simulation result by miss-positioning of a collimator in a constitution using a collimator relevant to Comparative Example, and FIG. 20 is a drawing showing change of imaging simulation result by miss-positioning of a collimator in a constitution using a collimator relevant to an embodiment of the present invention.

That is, FIG. 19 shows the one obtained by dividing FIG. 17C by FIG. 17A, and FIG. 20 shows the one obtained by dividing FIG. 18C by FIG. 18A.

As shown in FIG. 19, in the constitution using the collimator 26B relevant to Comparative Example, a streak orthogonal to a miss-positioning direction of the collimator 26B appears periodically in every other column.

On the other hand, as shown in FIG. 20, in the constitution using the collimator 26A relevant to an embodiment of the present invention, although a streak orthogonal to a miss-positioning direction of the collimator 26A appears at the edge of the planar source, a profile to change is irregular at other places. This is because of influence of using the Monte Carlo method in simulation.

In this way, it is understood that the constitution using the collimator 26A relevant to an embodiment of the present invention is stable against miss-positioning of the collimator 26.

In the constitution using the collimator 26B relevant to Comparative Example, because of appearance of a streak-like pattern, as shown in FIGS. 17B and 17C, by miss-positioning of the collimator 26B, ring-artifact appears in a size of about a pixel, as shown in FIG. 27 to be described later. By this artifact, a fine structure of a reconstructed image is lost, thus significantly deteriorating image quality.

Evaluation by an image of a uniform planar source becomes an index of whether artifact appears or not. It is because, in a region where a count number of radiation is nearly uniform, or in a region where change of the count number occurs in a scale sufficiently larger than a pixel size, artifact is conspicuous. Therefore, evaluation of artifact is enough to be performed using a radiation source with uniform distribution.

To make a matter simple, discussion will be given on a planar image obtained from the one having a uniform planar source and being parallel to the collimator 26. In addition, miss-positioning of the collimator 26 is assumed to be present only in one direction, and parallel to alignment of the detector 21.

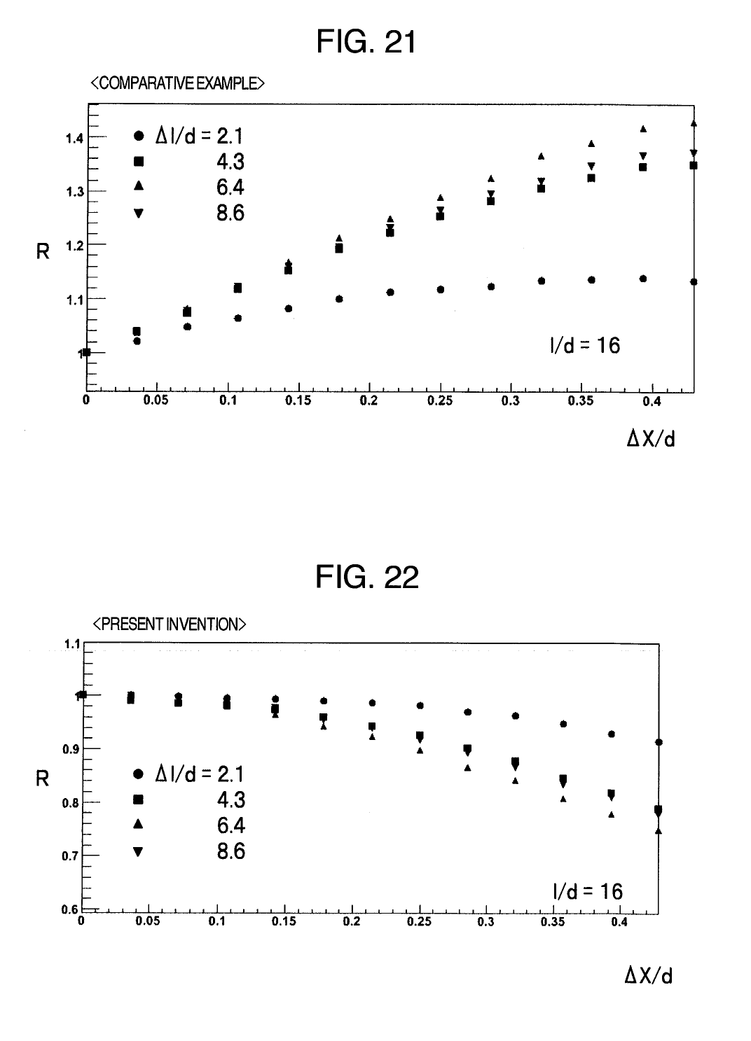

FIG. 21 shows how an image of the planar source changes with respect to miss-positioning of the collimator 26B, in the constitution using the collimator 26B relevant to Comparative Example.

FIG. 21 is a graph showing amount of change of a planar source image in a constitution using a collimator relevant to Comparative Example.

It should be noted that it is set that l/d=16 and t/d=0.3, and four kinds of values of 2.1, 4.3, 6.4 and 8.6 were used as for Δl/d.

Miss-positioning of the collimator 26B makes appearance of a peak and a bottom of a count number in every other column, in a column orthogonal to the miss-positioning direction (refer to FIG. 17A to 17C). A value obtained by averaging ratio of the count number of the peak and bottom of this count number in the detector group 21A, was adopted as R, which was used as the longitudinal axis of a graph. In addition, the one which was made dimensionless by dividing miss-positioning ΔX of the collimator 26B by d was adopted as the horizontal axis of the graph.

In the constitution using the collimator 26B relevant to Comparative Example, the case of no miss-positioning (ΔX=0) gives R=1. In addition, the larger Δl gives the larger change rate of R. This is because the larger Δl increases the more leaked radiation from the adjacent through-hole 27.

On the other hand, R in Δl/d=8.6 is smaller than R in Δl/d=6.4. This is because the case of Δl/d=8.6 and Δl/d=6.4 gives X/d>0.5, by which leaked radiation covers all the pixels detected, and acts in an erasing direction of the patterns of the peak and the valley. Although resolution also decreases accompanying with this, artifact is reduced as well. However, opening a space of Δl/d=8.6 or more gives significant deterioration of resolution, therefore consideration thereon may not be required.

FIG. 22 shows how an image of the planar source changes with respect to miss-positioning of the collimator 26A, in the constitution using the collimator 26A relevant to an embodiment of the present invention.

FIG. 22 is a graph showing amount of change of a planar source image in a constitution using a collimator relevant to an embodiment of the present invention.

It should be noted that, similarly as in FIG. 21, it is set that l/d=16 and t/d=0.3, and four kinds of values of 2.1, 4.3, 6.4 and 8.6 were used as for Δl/d.

A lattice stripe appears, even in a state that there is no miss-positioning of the collimator 26A, in the constitution using the collimator 26A relevant to an embodiment of the present invention (refer to FIGS. 18A to 18C). Here, the miss-positioning of the collimator 26A changes ratio of count number, in a column orthogonal to a miss-positioning direction. However, ratio of count number does not change, in a column in parallel to the miss-positioning direction.

Therefore, only ratio of count number in a column orthogonal to the miss-positioning direction was adopted as R. In addition, ratio R of count number in no miss-positioning (ΔX=0) was normalized to 1.

As shown in FIG. 22, in the constitution using the collimator 26A relevant to an embodiment of the present invention, the higher ΔX/d decreases R the more. In addition, similarly as in FIG. 21, because the case of Δl/d=8.6 and Δl/d=6.4 gives X/d>0.5, R in Δl/d=8.6 is smaller than R in Δl/d=6.4.

Within a range of |ΔX|≦d/2−X, that is, |ΔX|≦(d+t−T)/2−(Nd−t)Δl/l, in any of Δl/d, R changes. This is because not only leaked radiation but also shadow of the septa 28 contributes to change of the count number and the response function. However, change of R, as shown in FIG. 22, is within 10%. In practical use conditions, change of R is about 100 counts per one detector, which is within a range of a statistical error.

Therefore, in the constitution using the collimator 26A relevant to an embodiment of the present invention, the count number is within the statistical error and does not change, even when the collimator 26 moves within a range of |ΔX|≦(d+t−T)/2−(Nd−t)Δl/l.

Comparison will be given on the constitutions using the constitution using the collimator 26B relevant to Comparative Example and the constitution using the collimator 26A relevant to an embodiment of the present invention, in the case of Δl/d=4.3 in FIG. 21 and FIG. 22, as an example.

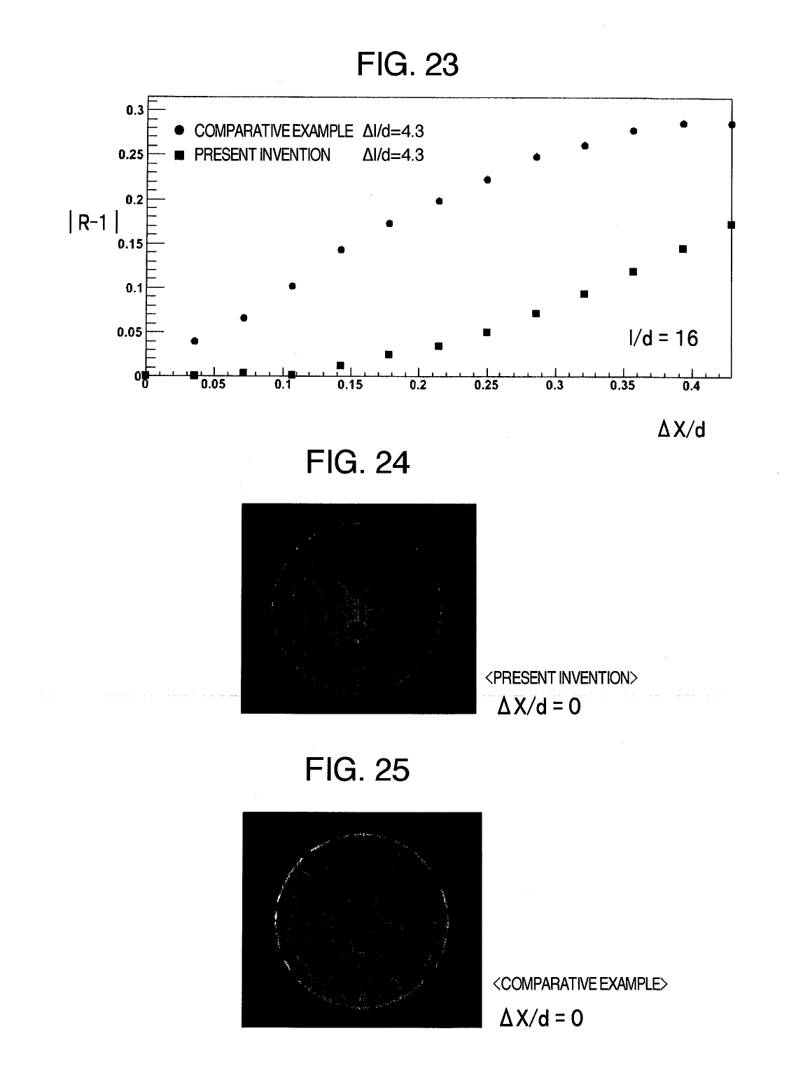

FIG. 23 is a graph showing amount of change of planar source image in Δl/d=4.3, in a constitution using the collimator relevant to an embodiment of the present invention, and a constitution using the collimator relevant to Comparative Example.

Absolute value of difference between ratio of the count number in no miss-positioning of the collimator 26 (ΔX=0) and 1, that is, |R−1|, is adopted as the longitudinal axis.

As shown in FIG. 23, the constitution using the collimator 26A relevant to an embodiment of the present invention has smaller change of |R−1| at the vicinity of ΔX=0, as compared with the constitution using the collimator 26B relevant to Comparative Example, that is, stable against miss-positioning of the collimator 26.

Up to now, discussion has been made only on miss-positioning of the collimator 26 in one direction. However, this may be the same for miss-positioning in two directions. In addition, as for slight rotation of the collimator 26, it is equivalent to miss-positioning in two directions, when viewed locally. Therefore, a stable constitution with respect to miss-positioning can be also stable with respect to rotation.

### <Resolution>

Because images of FIGS. 18A to 18C have specific patterns, the FBP method cannot be applied. In addition, as shown in FIG. 16, a view from the detector 21 under the septa 28 is separated. However, in the iterative reconstruction method using the response function, an image can be reconstructed. In addition, use of the response function in consideration of the aforementioned separated view provides little decrease in resolution. It should be noted that irradiation of a uniform planar source provides an image with a periodic pattern, and thus provides the response function having a complicated shape.

Resolution in the constitution using the collimator 26A relevant to an embodiment of the present invention (refer to FIG. 8 and FIG. 10)) and the constitution using the collimator 26B relevant to Comparative Example (refer to FIG. 9 and FIG. 11) was compared by simulation. It should be noted that simulation was performed under condition of l/d=18.6, Δl/d=5.7, T/d=0.07, t/d=0.3, and ΔX=0. In each constitution, the response function was determined, and by utilization of the response function a sinogram was prepared from digital phantom. It should be noted that statistical fluctuation is not taken into consideration. And, FIG. 24 and FIG. 25 show results of reconstitution of the resultant sinogram, by the OSEM method.

FIG. 24 is an example of simulation result of image reconstitution in a constitution using the collimator relevant to an embodiment of the present invention. FIG. 25 is an example of simulation result of image reconstitution in a constitution using the collimator relevant to Comparative Example.

In this way, it is understood that there is no difference of resolution between two constitutions, when there is no miss-positioning of the collimator 26 (ΔX=0).

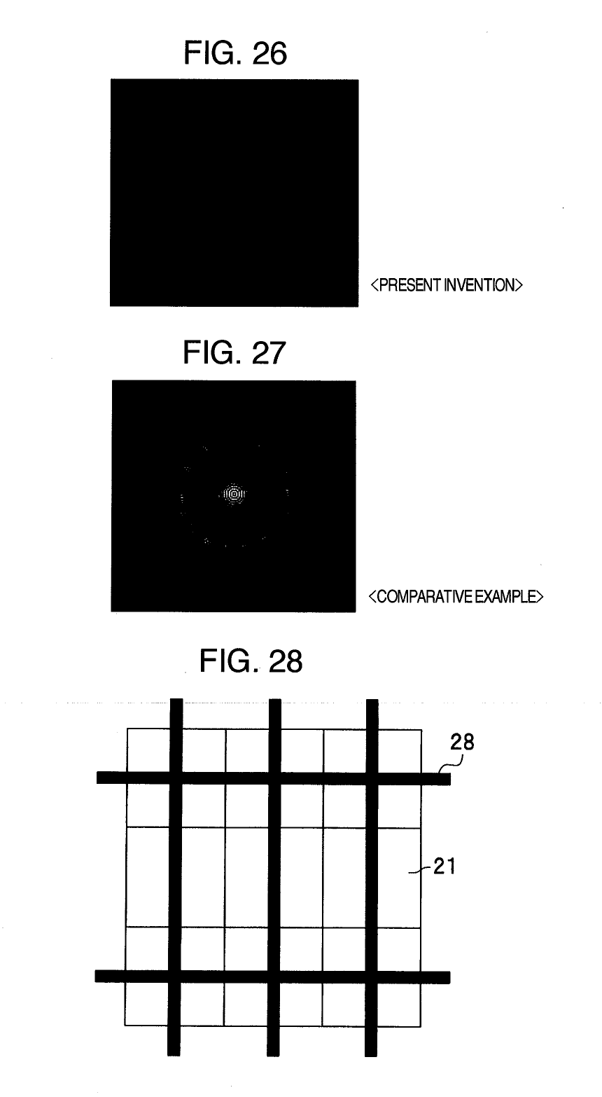

Next, absolute value of difference between a reconstructed image obtained in ΔX/d=0, and a reconstructed image obtained in ΔX/d=0.07 is shown in FIG. 26 and FIG. 27.

FIG. 26 is a drawing showing change of simulation result of image reconstitution by miss-positioning of a collimator in a constitution using the collimator relevant to an embodiment of the present invention, and FIG. 27 is a drawing showing change of simulation result of image reconstitution by miss-positioning of a collimator in a constitution using the collimator relevant to Comparative Example.

It should be noted that scales of FIG. 26 and FIG. 27 are unified.

It is well understood that, in the constitution using the collimator 26B relevant to Comparative Example, as shown in FIG. 27, stripe-like artifact appears. Absolute value of difference between the reconstructed image obtained in ΔX/d=0, and the reconstructed image obtained in ΔX/d=0.07 is about 15%.

On the other hand, in the constitution using the collimator 26A relevant to an embodiment of the present invention, as shown in FIG. 26, artifact is little observed, and absolute value of difference between the reconstructed image obtained in ΔX/d=0, and the reconstructed image obtained in ΔX/d=0.07 is about 3%, which is about ⅕ of Comparative Example. This is far smaller than statistical error (10%), under condition of 100 counts per detector. It should be noted that ΔX/d=0.07 is equivalent to miss-positioning of ΔX=0.07 mm, in the detector 21 having a pixel size of about d=1 mm. Miss-positioning of this degree is unavoidable, in consideration of manufacturing precision of the collimator 26.

Therefore, in the constitution using the collimator 26B relevant to Comparative Example, appearance of artifact is unavoidable, however, in the constitution using the collimator 26A relevant to an embodiment of the present invention, it can be decreased in a large degree.

In addition, as in the constitution using the collimator 26A relevant to an embodiment of the present invention, arrangement of the collimator 26 at the vicinity of the center of the detector 21 decreases the sensitivity of the detector 21, caused by shadow of the collimator 26. However, in practical, decrease in sensitivity may be only a little, or sensitivity may rather increase in some cases. It is because there is generally a gap between the detector 21 and the detector 21 in the pixel-type detector 21. Therefore, that region is insensitive (corresponding to the insensible field 31 of FIG. 15). When the septa 28 is arranged on the boundary line 32 between this detector 21 pair or at the vicinity thereof, in a plane view from a direction perpendicular to the detector plane, leaked radiation distributes at that insensible field 31. Therefore, these radiations are not detected. On the other hand, in the present invention, because these leaked radiations distribute on the detector 21, all of them are detected. In this way, due to trade off relation between shadow of the collimator 26 and leaked radiation, sensitivity can be maintained.

Therefore, in the SPECT device 1 having the constitution using the collimator 26A relevant to an embodiment of the present invention, the collimator 26 having conventional manufacturing precision can be used. In addition, also in positioning of the collimator 26, lower precision than that of the constitution using the collimator 26B relevant to Comparative Example may be allowed. In this way, time required for positioning can be shortened. In addition, precision in positioning of the collimator 26, even in the detector 21 with a pixel size of 1 mm, may be about 0.1 mm, which is a practical value. In addition, because of being more stable against miss-positioning of the collimator 26 in measurement, than that of the constitution using the collimator 26B relevant to Comparative Example, an image having less artifact can be obtained.

Still more, by using a method for recording count number of each detector 21 in measurement, and combining the count number of a plurality of adjacent detectors 21 after measurement by off-line, number of the detector 21 to be combined can be changed freely. In this way, an image can be reconstructed in various resolutions after measurement. In this SPECT device 1, resolution, and minimal count number required in imaging can be selected freely, without changing constitution of a device.

In addition, this constitution is effective in using a collimator for medium energy or a collimator for high energy. The higher energy of radiation to be used increases penetrating power of a substance the higher, and thus thickness of the septa 28 is required to increase, accompanying with which, dead space increases, and sensitivity decreases. To prevent this, it is general to use the one with a large size of the through-hole 27. However, in the present invention, by arranging a plurality of the detectors 21 in one through-hole 27, resolution can be maintained, even when hole diameter is increased. Therefore, it is effective for imaging using y-radiation having high energy.

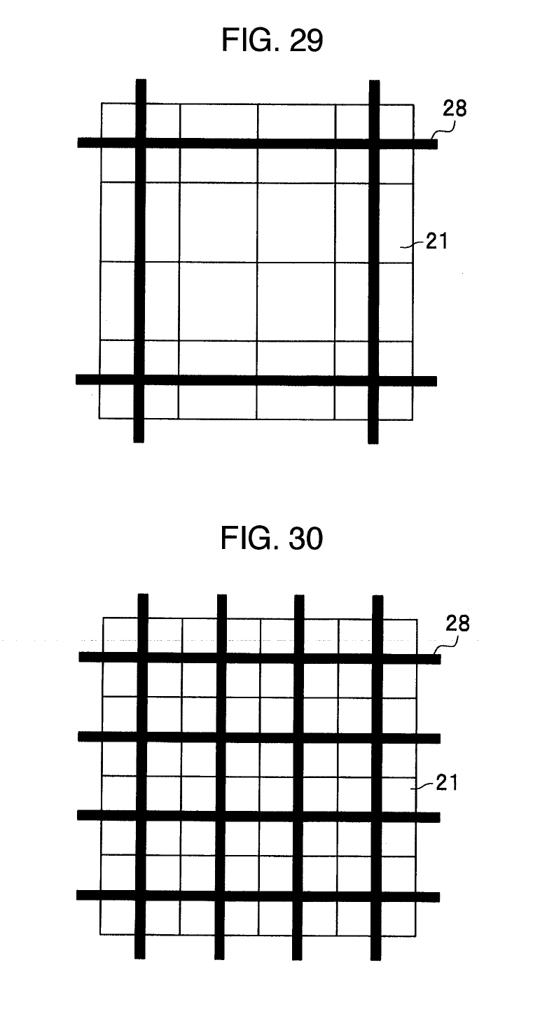

It should be noted that the collimator 26A has been explained as a constitution containing four sets of the detectors 21 per one through-hole 27A, however, it is not limited to this case, and number of the detector 21 contained within the through-hole 27 may be arbitrary, as long as the septa 28 passes the center of the detector 21, and is orthogonal to the end part of the detector 21. For example, as shown in FIG. 28, it may be a constitution containing two sets of the detectors 21, or it may be a constitution containing nine sets of the detectors 21, as shown in FIG. 29.

### <Modification Examples>

It is a general constitution where the through-hole 27 of the collimator 26 and the detector 21 are arranged so that the through-hole 27 corresponds one-to-one with the detector 21. In this constitution, in the case of miss-positioning of the collimator 26 in parallel to alignment of the detector 21, change of the response function is the same for each of the detectors 21.

Therefore, in the case of miss-positioning of the collimator 26 in parallel to alignment of the detector 21, reconstitution of an image, by the response function in “no miss-positioning” of the collimator 2, does not provide artifact, although decreases resolution.

However, addition of rotation to miss-positioning of the collimator 26 changes a way of miss-positioning of the collimator 26 depending on position of the detector 21, which makes appear the non-uniformity of sensitivity in a large scale, thus forming moiré. This moiré never deteriorates image quality in a large degree, however, to provide a tomography image with higher picture quality, it is necessary to remove moiré. In particular, the smaller size of the detector 21 makes influence by rotation the more significant. It is because even a small rotation changes positional relation between the collimator 26 and the detector 21 largely. In recent years, the detector 21 having a size of about 1 mm has been developed, which has increased necessity to remove moiré caused by rotation.

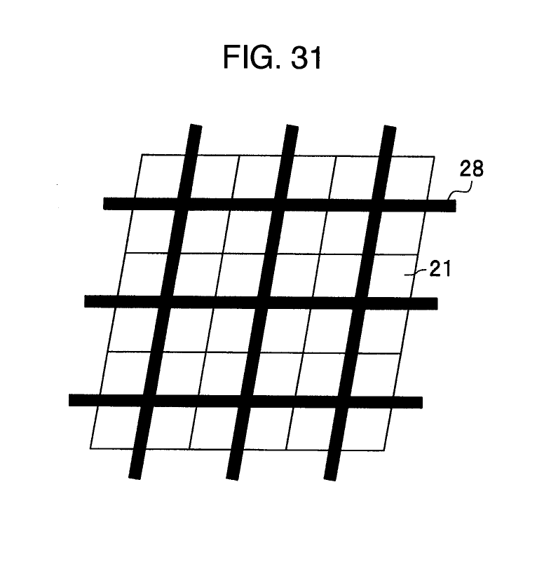

Also in this case, there is a constitution where the response function is stable against miss-positioning of the collimator 26. As shown in FIG. 30, it is general that the detector 21 and the through-hole 27 surrounded by the septa 28 is rectangle, however, the case of a parallelogram, as shown in FIG. 31, is similar, as well.

Length of one side of the through-hole 27 of one collimator 26 is represented by L. In the previous example, L=Nd−t. In the case where length of an arbitrary line connecting the septa 28 and the boundary line 32 between this detector 21 pair is always LΔl/l+(T−t)/2 or longer, in a plane view from a direction perpendicular to the detector plane, leaked radiation is restricted within the same pixel, and is stable against miss-positioning of the collimator 26.

In this case, it also provides stability against rotation of the collimator 26, and is also possible to prevent moiré.

In addition, the septa 28 is arranged in parallel to alignment of the detector 21. It is because of aiming at uniform sensitivity of each detector 21. In these constitutions shown in FIG. 30 and FIG. 31, an image can be reconstructed by the FBP method and the iterative reconstruction method.

In addition, in the SPECT device 1 provided with the collimator 26 having the detector 21 with an arbitrary shape, and the through-hole 27 with an arbitrary shape, it is enough that distance between the boundary line 32 between a detector 21 pair and the septa 28, in a plane view from a direction perpendicular to the detector plane and, is LΔl/l+(T−t)/2 or longer, in a plane view from a direction perpendicular to the detector plane. In this way, leaked radiation is restricted in the same pixel. However, in an arbitrary shape of the through-hole 27, L is defined as the maximum width of the through-hole 27. In addition, distance between the septa 28 and the boundary line 32 between this detector 21 pair, perpendicular each other, may be the above value or shorter. In this way, area of a distribution region of the leaked radiation on the detector 21 can be maintained constant, even in a little miss-positioning of the collimator 26. Such setting is enough that length of an arbitrary line drawn down from the top of the through-hole 27 to the boundary line 32 between the detector 21 pair is always LΔl/l+(T−t)/2 or longer. It is good that length of an arbitrary line drawn down from the top of the detector 21 to the septa 28 is always LΔl/l+(T−t)/2 or longer.

Even when the collimator 26 moves within the above range, distribution area of the leaked radiation on one detector 21 is constant.

The above description was provided on Examples, however, it is apparent to those skilled in the art that the present invention should not to be limited to these, and various changes and modifications are possible within a range of the spirit and claims of the present invention.

### REFERENCE SIGNS LIST

1 SPECT device (nuclear medicine diagnostic device)
10 Gantry
11A, 11B Camera (radiation imaging device)
12 Data processing device (tomography image information preparation device)
13 Display device
14 Bed
15 Subject
21 Detector
21A, 21B, 21C, 21D, 21E Detector group
21f Incident plane
21g Scintillator
21h Photodiode
22a, 22b, 22c, 22d, 22e, 22f Electrode
22c Common electrode
23 Detector substrate
24 ASIC substrate
25 ASIC (radiation measuring circuit)
26, 26A, 26B Collimator
27, 27A, 27B Through-hole
28, 28A, 28B Septa
29 Light shielding, γ-radiation and electromagnetic shield
29 Boundary plane
31 Insensible field
32 Boundary line

1. A nuclear medicine diagnostic device comprising:
a pixel-type detector group where detectors are arrayed having a spread as a plane, and each detector constitutes each pixel;
a radiation measuring circuit which reads out a detection signal from the detector group; and
a collimator partitioned by a septa, and arrayed with a plurality of through-holes which are extending in a direction perpendicular to the detector plane; characterized by comprising:
a radiation imaging device which acquires incident position information of radiation emitted from a body of a subject mounted on a bed for each detector by:
setting the size of the through-hole such that a plurality of the pixels are arrayed in a plane view from the direction perpendicular to the detector plane;
arranging the septa in displacing from a boundary line between the detector pair in a plane view from the direction perpendicular to the detector plane;
further arranging the septa so as to be orthogonal to the boundary line between the detector pair in a plane view from the direction perpendicular to the detector plane;
arranging the top of the through-hole in a plane view from the direction perpendicular to the detector plane in displacing from the boundary line of the detector pair; and
arranging the top of the detectors in a plane view from the direction perpendicular to the detector plane so as to be visible through the through-hole; and
a tomography image information preparation device which forms picture image information using a response function, by acquiring a detection signal from the detector.

2. The nuclear medicine diagnostic device according to claim 1, characterized in that:
provided that height of the collimator is l, distance between the collimator and the detector is Δl, thickness of the septa is t, maximum width of the through-hole is L, and width of an insensible field between the detectors is T;
length of an arbitrary line connecting the septa in a plane view from the direction perpendicular to the detector plane and the boundary line between the detector pair is always LΔl/l+(T−t)/2 or longer;
the length may be arbitrary, when the septa in a plane view from the direction perpendicular to the detector plane is orthogonal to the boundary line between the detector pair;
length of a line drawn down from the top of the through-hole in a plan view from the direction perpendicular to the detector plane to the boundary line between the detector pair is always LΔl/l+(T−t)/2 or longer; and
length of a line drawn down from the top of the detector in a plane view from the direction perpendicular to the detector plane to the septa is always LΔl/l+(T−t)/2 or longer.

3. (canceled)

4. A nuclear medicine diagnostic device having the radiation imaging device according to claim 1, characterized in that:
provided that the through-hole is rectangle, and maximum width of the through-hole is L;
a distance between centers of the detectors is d; and
the top of the septa in a plane view from the direction perpendicular to the detector plane is arranged within (d+t−T)/2−LΔl/l from the center of the detector.

5. A nuclear medicine diagnostic device having the radiation imaging device according to claim 2, characterized in that:
provided that the through-hole is rectangle, and maximum width of the through-hole is L;
the top of the septa in a plane view from the direction perpendicular to the detector plane is arranged within (d+t−T)/2−LΔl/l from the center of the detector.

6. A nuclear medicine diagnostic device having the radiation imaging device according to claim 1, characterized in that:
the top of the septa in a plane view from the direction perpendicular to the detector plane is arranged at the center of the detector.

7. A nuclear medicine diagnostic device having the radiation imaging device according to claim 2, characterized in that:
the top of the septa in a plane view from the direction perpendicular to the detector plane is arranged at the center of the detector.

8. A nuclear medicine diagnostic device having the radiation imaging device according to claim 1, characterized in that:
the through-hole is rectangle, and the top of the septa in a plane view from the direction perpendicular to the detector plane is arranged at the center of the detector.

9. A nuclear medicine diagnostic device having the radiation imaging device according to claim 2, characterized in that:
the through-hole is rectangle, and the top of the septa in a plane view from the direction perpendicular to the detector plane is arranged at the center of the detector.
